(19) United States
(12) Patent Application Publication
(10) Pub. No.: US 2025/0195540 A1
(43) Pub. Date: Jun. 19, 2025

(54) Use of dehydroepiandrosterone (DHEA) in preparation of drug for preventing and treating ophthalmic myopia, dosage forms of DHEA, and preparation methods of dosage forms

(71) Applicant: Zhongshan Ophthalmic Center of Sun Yat-sen University, Guangzhou (CN)

(72) Inventors: Shida CHEN, Guangzhou (CN); Ping LIAN, Guangzhou (CN); Xiujuan ZHAO, Guangzhou (CN); Lin LV, Guangzhou (CN)

(21) Appl. No.: 19/066,888

(22) Filed: Feb. 28, 2025

Related U.S. Application Data
(63) Continuation of application No. PCT/CN2023/110037, filed on Jul. 28, 2023.

(30) Foreign Application Priority Data
Aug. 29, 2022 (CN) 202211038346.1

(51) Int. Cl. A61K 31/5685; A61K 9/00; A61K 9/06; A61K 9/08; A61K 47/02; A61K 47/18; A61K 47/38; A61K 47/44; A61P 27/10

(52) U.S. Cl. CPC A61K 31/5685; A61K 9/0048; A61K 9/06; A61K 9/08; A61K 47/02; A61K 47/186; A61K 47/38; A61K 47/44; A61P 27/10

(57) ABSTRACT

The present disclosure discloses a use of dehydroepiandrosterone (DHEA) in preparation of a drug for preventing and treating ophthalmic myopia. The drug is preferably for external use. The dosage form of the drug is an eye drop, an eye ointment, or a gel. The present disclosure also discloses the component of the DHEA drug in dosage form of eye drop, eye ointment, or gel, and a respective preparation method thereof. Experimental results have shown that the pharmaceutical formulations of the present disclosure can be effectively used for prevention and control of myopia, and is economical, safe, and effective.

### CROSS REFERENCE TO RELATED APPLICATIONS

The present application is a continuation of International Application No. PCT/CN2023/110037, filed on Jul. 28, 2023, which claims priority from Chinese Application No. 202211038346.1 filed on Aug. 29, 2022, all of which are hereby incorporated herein by reference.

### TECHNICAL FIELD

The present disclosure relates to the technical field of ophthalmic drugs, specifically to a use of dehydroepiandrosterone (DHEA) in preparation of a drug for preventing and treating ophthalmic myopia, dosage forms of the DHEA, and preparation methods of the dosage forms.

### BACKGROUND

The population suffer from myopia has been increasing and has also become younger and younger. However, there is currently no definite and effective method for preventing and treating myopia. Some researches have shown that increasing the amount of time spent outdoors under sun light, applying atropine eye drops, and wearing orthokeratology lenses are currently promising interventions for myopia control. However, outdoor activities can only slow down the progression of myopia and may require multi-sided coordination from children, parents and schools, and time spent on close reading needs to be reduced, which are challenging for a long time. Additionally, patients using atropine eye drops will undergo a rebound of myopia after discontinuing drugs for a certain period, which is manifested as myopia progression. Moreover, atropine eye drops lead to high incidence rates of systemic and local adverse reactions. Patients who wear orthokeratology lenses is also required to continue the wearing every day, which is difficult to stick to. In addition, patients who wear orthokeratology lenses are prone to the risk of corneal damage, infection, or damage to the ophthalmic surface environment, and easily undergo a rebound of myopia after stopping wearing. Therefore, there is an urgent need to develop appropriate ophthalmic drugs for preventing and treating myopia.

### SUMMARY

The present disclosure aims to overcome at least one of the defects (deficiencies) of the prior art. In one aspect, the present disclosure provides a use of dehydroepiandrosterone (DHEA) in preparation of a drug for preventing and treating ophthalmic myopia.

A second object of the present disclosure is to provide a DHEA-based eye drop and a preparation method thereof.

A third object of the present disclosure is to provide a DHEA-based eye ointment and a preparation method thereof.

A fourth object of the present disclosure is to provide a DHEA-based gel and a preparation method thereof.

According to the present disclosure, a use of DHEA in preparation of a drug for preventing and treating myopia is provided.

Dehydroepiandrosterone, also known as DHEA, is chemically known as 3-β-hydroxyandrost-5-en-17-one, with a molecular formula of C₁₉H₂₈O₂ and a molecular weight of 288.41. DHEA is an adrenal hormone precursor secreted by the zona reticularis of the human adrenal cortex, which exists mostly in the sulphated form (DHEA-s) in the blood, and they are the key precursors for the synthesis of estrogen and testosterone in the human body. Normally, the amount of DHEA secreted in the human body gradually declines with age. Currently, DHEA, as an oral drug and a health care drug, has the effects of regulating obesity, preventing diabetes, resisting carcinogenic and viral infection, improving memory, immune response, and stress response, and alleviating tension. However, there are still few studies on eyes.

Through clinical and animal experimental studies, the present inventor has found that DHEA is involved in the occurrence mechanisms of various ophthalmic lesions, especially myopia, and that DHEA can reduce the growth of axial length (AL) in animal models when applied locally to eyes and thus has the effect of preventing and treating myopia.

The drug using DHEA is preferably for external use.

The topical and external administration to eyes is also a convenient and non-invasive ophthalmic administration route for treating eye diseases, and is more in line with the needs of users.

The dosage form of the drug is preferably an eye drop, an eye ointment, or a gel.

Provided is a DHEA-based eye drop according to the present disclosure, including the following pharmaceutical components in parts by weight: 8 to 10 parts of hydroxypropyl methylcellulose; 24 to 28 parts of boric acid; 1 to 5 parts of borax; 0.05 to 0.1 part of benzalkonium chloride; 0.04 to 8 parts of DHEA; 0.001 to 0.1 part of a pH-adjusting agent; and 0.001 to 0.1 part of an osmolarity adjusting agent.

A preparation method of the DHEA-based eye drop described above including the following steps.
S1) weighing 8 to 10 parts by weight of the hydroxypropyl methylcellulose, 24 to 28 parts by weight of the boric acid, 1 to 5 parts by weight of the borax, 0.05 to 0.1 parts by weight of the benzalkonium chloride, and 1,350 to 2,150 parts by weight of purified water to prepare a blank solvent;
S2) weighing 0.04 to 8 parts by weight of the DHEA and 400 parts by weight of the blank solvent to prepare a DHEA solution; and
S3) adding the pH-adjusting agent and the osmolarity adjusting agent to the DHEA solution, respectively, to adjust the pH and osmolarity of the DHEA solution to 6.2 to 7.2 and 280 mOsm/L to 310 mOsm/L respectively to obtain the DHEA-based eye drop.

Further, in the step S1, the blank solvent is preferably prepared by the following specific steps.
S11) weighing 8 to 10 parts by weight of the hydroxypropyl methylcellulose, and dissolving the hydroxypropyl methylcellulose in 350 to 450 parts by weight of the purified water to produce a first solution;
S12) weighing 24 to 28 parts by weight of the boric acid and 1 to 5 parts by weight of the borax, and dissolving the boric acid and the borax in the first solution to produce a second solution; and
S13) weighing 0.05 to 0.1 part by weight of the benzalkonium chloride, dissolving the benzalkonium chloride in the second solution, and diluting with 1,300 to 1,700 parts by weight of the purified water to a specified volume or concentration to produce the blank solvent.

In the step S2, the DHEA solution is preferably prepared by the following specific step:
weighing 0.04 to 8 parts by weight of the DHEA and 400 parts by weight of the blank solvent, and dispersing the DHEA evenly in the blank solvent with a high-shear emulsifying machine to produce the DHEA solution.

A rotational speed of the high-shear emulsifying machine may be 1,000 r/min.

Concentration percentage of the DHEA is calculated by the ratio of the parts by weight of the DHEA to 400 parts by weight of the blank solvent, namely=parts by weight of the DHEA/400 parts by weight of the blank solvent×100%.

When the DHEA is of 8 parts, the concentration of the DHEA solution obtained is 2%.

When the DHEA is of 4 parts, the concentration of the DHEA solution obtained is 1%.

When the DHEA is of 2 parts, the concentration of the DHEA solution obtained is 0.5%.

When the DHEA is of 0.4 part, the concentration of the DHEA solution obtained is 0.1%.

When the DHEA is of 0.2 part, the concentration of the DHEA solution obtained is 0.05%.

When the DHEA is of 0.04 part, the concentration of the DHEA solution obtained is 0.01%.

According to the above preparation relationship:

When a DHEA solution with a concentration of 1% is prepared from a DHEA solution with a concentration of 2%, 1 part by weight of the blank solvent is added to each 1 part by weight of the DHEA solution with the concentration of 2%.

When a DHEA solution with a concentration of 0.5% is prepared from a DHEA solution with a concentration of 1%, 1 part by weight of the blank solvent is added to each 1 part by weight of the DHEA solution with the concentration of 1%.

When a DHEA solution with a concentration of 0.1% is prepared from a DHEA solution with a concentration of 0.5%, 4 parts by weight of the blank solvent are added to each 1 part by weight of the DHEA solution with the concentration of 0.5%.

When a DHEA solution with a concentration of 0.05% is prepared from a DHEA solution with a concentration of 0.1%, 1 part by weight of the blank solvent is added to each 1 part by weight of the DHEA solution with the concentration of 0.1%.

When a DHEA solution with a concentration of 0.01% is prepared from a DHEA solution with a concentration of 0.05%, 4 parts by weight of the blank solvent are added to each 1 part by weight of the DHEA solution with the concentration of 0.05%.

In the S3, the pH-adjusting agent is added to adjust a pH of the eye drop to 6.2 to 7.2. The pH-adjusting agent may be disodium phosphate, or monosodium phosphate, or a mixture of disodium phosphate and monosodium phosphate. The osmolarity adjusting agent is added to adjust an osmolarity to 280 mOsm/L to 310 mOsm/L. The osmolarity adjusting agent may be sodium chloride.

A DHEA-based eye ointment is provided according to the present disclosure, including the following pharmaceutical components in parts by weight: 5 to 10 parts of DHEA; 25 to 50 parts of lanolin; 12 to 25 parts of sterile liquid paraffin; and 850 to 1,000 parts of yellow Vaseline.

A preparation method of the DHEA-based eye ointment described above is provided, including the following steps.
S1) preparation of an eye ointment matrix: including weighing 25 to 50 parts by weight of the lanolin and 850 to 1,000 parts by weight of the yellow vaseline, mixing the lanolin and the yellow Vaseline, and heating for melting; and then adding 12 to 25 parts by weight of the sterile liquid paraffin, thoroughly mixing, and cooling to produce the eye ointment matrix;
S2) weighing 5 to 10 parts by weight of the DHEA, and dissolving the DHEA in 4 to 8 parts by weight of the sterile water for injection to produce a mixed solution; and
S3) adding the mixed solution obtained in the S2 to the eye ointment matrix obtained in the S1, and thoroughly stirring to produce the DHEA-based eye ointment.

A DHEA-based gel is provided according to the present disclosure, including the following pharmaceutical components in parts by weight: 0.5 to 2 parts of DHEA; 1 to 5 parts of a thickening agent; 0.8 to 1.5 part of an isotonic agent; 0.001 to 0.05 part of a bacteriostatic agent; 0.001 to 0.005 part of a pH-adjusting agent; and 0.2 to 1 part of a humectant.

A preparation method of the DHEA-based gel described above is provided, including the following steps.
S1) weighing 1 to 5 parts by weight of the thickening agent, dissolving the thickening agent in 5 to 10 parts by weight of sterile water for injection, and then adjusting a pH with the pH-adjusting agent to 6.5 to 8.0 to produce a solution 1; and
S2) weighing and adding the 0.5 to 2 parts by weight of the DHEA, the 0.8 to 1.5 parts by weight of the isotonic agent, the 0.001 to 0.05 part by weight of the bacteriostatic agent, and the 0.2 to 1 part by weight of the humectant to the solution 1, supplementing sterile water for injection until a resulting solution is of 100 parts by weight in total, and thoroughly mixing to produce the DHEA-based gel.

In the present disclosure, the thickening agent may be carbomer, the isotonic agent may be sodium chloride, the bacteriostatic agent may be benzalkonium bromide, the pH-adjusting agent may be phosphoric acid, and the humectant may be sodium hyaluronate. Specifically, the pH-adjusting agent is used to adjust the pH of the DHEA-based gel to 7.

Further, a use of the DHEA-based eye drop described above, the DHEA-based eye ointment described above, and/or the DHEA-based gel described above in preparation of a drug for preventing and treating ophthalmic myopia is provided. The ophthalmic myopia includes true myopia and pseudomyopia.

Compared with the prior art, the present disclosure has the following beneficial effects.

The present disclosure discloses a use of DHEA in preparation of a drug for preventing and treating ophthalmic myopia. The existing drugs for treating ophthalmic myopia are mainly atropine. Compared with the existing ophthalmic formulations, the present application innovatively adopts DHEA to prepare a drug for ophthalmic myopia. Moreover, a large number of experimental results have shown that the ophthalmic formulations prepared in the present application have the beneficial effects of safety, economy, effectiveness, and small irritation, and are more in line with the needs of users. The present disclosure also provides preparation methods of a DHEA-based eye drop, a DHEA-based eye ointment, a DHEA-based gel, and a DHEA-based liquid temperature-sensitive gel. It has been proved through experimental results that the DHEA-based ophthalmic formulations exhibit a significant therapeutic effect in the prevention and treatment of ophthalmic myopia, and have strong practical performance.

### BRIEF DESCRIPTION OF THE DRAWINGS

FIG. 1 shows the changes in ALs after the application of DHEA-based eye drops;

FIG. 2 shows the changes in diopters after the application of DHEA-based eye drops;

FIG. 3 shows the changes in ALs after the application of a DHEA-based eye ointment (Oint) and a DHEA-based gel (Gel); and

FIG. 4 shows the changes in diopters after the application of a DHEA-based eye ointment (Oint) and a DHEA-based gel (Gel).

### DETAILED DESCRIPTION

### Example 1

In this example, a DHEA-based eye drop was disclosed, including the following pharmaceutical components: DHEA, a pH-adjusting agent, an osmolarity adjusting agent, and a blank solvent. Specifically, a preparation method of the DHEA-based eye drop in this example was as follows.

In step S1, a blank solvent was prepared. Specifically, 9 parts by weight of hydroxypropyl methylcellulose were weighed and dissolved in 400 parts by weight of purified water to produce a first solution; then 26 parts by weight of boric acid and 3 parts by weight of borax were weighed and added to the first solution, and stirring was conducted for complete dissolution to produce a second solution; 0.075 part by weight of benzalkonium chloride was weighed and added to the second solution, stirring was conducted for complete dissolution, and finally 1,500 parts by weight of purified water were added for diluting to a specified volume or concentration to produce the blank solvent.

In step S2, DHEA solutions with concentrations of 0.01% to 2% were prepared. Specific preparation processes were as follows:
S21) Preparation of a DHEA solution with a concentration of 2%: 8 parts by weight of a DHEA were weighed, 400 parts by weight of the blank solvent were added for dissolution, and even dispersion was allowed with a high-shear emulsifying machine at 1,000 r/min to produce the DHEA solution with the concentration of 2%.
S22) Preparation of a DHEA solution with a concentration of 1%: 1 part by weight of the DHEA solution with the concentration of 2% was weighed, 1 part by weight of the blank solvent was added, and even dispersion was allowed with a high-shear emulsifying machine at 1,000 r/min to produce the DHEA solution with the concentration of 1%.
S23) Preparation of a DHEA solution with a concentration of 0.5%: 1 part by weight of the DHEA solution with the concentration of 1% was weighed, 1 part by weight of the blank solvent was added, and even dispersion was allowed with a high-shear emulsifying machine at 1,000 r/min to produce the DHEA solution with the concentration of 0.5%.
S24) Preparation of a DHEA solution with a concentration of 0.1%: 1 part by weight of the DHEA solution with the concentration of 0.5% was weighed, 4 parts by weight of the blank solvent were added, and even dispersion was allowed with a high-shear emulsifying machine at 1,000 r/min to produce the DHEA solution with the concentration of 0.1%.
S25) Preparation of a DHEA solution with a concentration of 0.05%: 1 part by weight of the DHEA solution with the concentration of 0.1% was weighed, 1 part by weight of the blank solvent was added, and even dispersion was allowed with a high-shear emulsifying machine at 1,000 r/min to produce the DHEA solution with the concentration of 0.05%.
S26) Preparation of a DHEA solution with a concentration of 0.01%: 1 part by weight of the DHEA solution with the concentration of 0.05% was weighed, 4 parts by weight of the blank solvent were added, and even dispersion was allowed with a high-shear emulsifying machine at 1,000 r/min to produce the DHEA solution with the concentration of 0.01%.

In step S3, the pH-adjusting agent and the osmolarity adjusting agent were added to each DHEA solution prepared to adjust a pH and an osmolarity of the DHEA solution to 6.2 to 7.2 and 280 to 310 mOsm/L respectively to produce the DHEA-based eye drop.

Specifically, in this example, pH values and osmolarity of DHEA-based eye drops with different concentrations were shown in Table 1 below.

TABLE 1

| Concentration | pH | Osmolarity |
|---|---|---|
| 0.01% DHEA | 6.9 | 295 |
| 0.05% DHEA | 7.0 | 298 |
| 0.1% DHEA | 7.1 | 297 |
| 0.5% DHEA | 7.0 | 297 |
| 1% DHEA | 7.04 | 298 |
| 2% DHEA | 7.06 | 295 |

Specifically, in this example, a main function of the pH-adjusting agent added is to keep a pH of an eye drop basically consistent with the pH of the human tears, which allows little irritation. A main function of the osmolarity adjusting agent added is to keep an osmolarity of an eye drop close to the osmolarity of the human tears.

### Example 2

This example was different from Example 1 in that: in the step S2, according to the following relationship: concentration percentage of DHEA=parts by weight of a DHEA/400 parts by weight of a blank solvent×100%, required parts by weight of the DHEA and the blank solvent were weighed, and then the DHEA was dispersed evenly in the blank solvent with a high-shear emulsifying machine to prepare a DHEA solution with a concentration of 0.01% to 2%.

Specifically, in this example, preparation parameters for DHEA-based eye drops with different concentrations were shown in Table 2 below.

TABLE 2

| Concentration | DHEA | Blank solvent |
|---|---|---|
| 0.01% DHEA | 0.04 parts by weight | 400 parts by weight |
| 0.05% DHEA | 0.2 parts by weight | 400 parts by weight |
| 0.1% DHEA | 0.4 parts by weight | 400 parts by weight |
| 0.5% DHEA | 2 parts by weight | 400 parts by weight |
| 1% DHEA | 4 parts by weight | 400 parts by weight |
| 2% DHEA | 8 parts by weight | 400 parts by weight |

### Example 3

In this example, a DHEA-based eye ointment was disclosed, including the following pharmaceutical components in parts by weight: 5 parts by weight of DHEA; 25 parts by weight of lanolin; 12.5 parts by weight of sterile liquid paraffin; 953.5 parts by weight of yellow Vaseline; and 4 parts by weight of sterile water for injection, with 1,000 parts in total.

The DHEA-based eye ointment was prepared through the following method.

A preparation method of the DHEA-based eye ointment included the following steps.

In step S1, an eye ointment matrix is prepared. Specifically, 25 parts by weight of lanolin and 953.5 parts by weight of yellow Vaseline were weighed, mixed, and heated for melting. Then 12.5 parts by weight of sterile liquid paraffin were added, and then thorough mixing and cooling were conducted to produce the eye ointment matrix.

In step S2, 5 parts by weight of a DHEA were weighed and dissolved in 4 parts by weight of sterile water for injection to produce a mixed solution.

In step S3, the mixed solution obtained in the step S2 was added to the eye ointment matrix obtained in the step S1 to produce a mixture. The mixture was thoroughly stirred and aseptically dispensed to produce the DHEA-based eye ointment.

Specifically, in the step S1, the lanolin and the yellow Vaseline were heated at 100° C. to achieve the purpose of sterilization, and the cooling was conducted to 80° C. In the step S3, the mixed solution was added to the eye ointment matrix under vigorous stirring.

A mass percentage of DHEA in the DHEA-based eye ointment obtained in this example was 0.5%.

### Example 4

In this example, a DHEA-based gel was disclosed, including the following pharmaceutical components in parts by weight: 0.5 to 2 parts by weight of DHEA; 1 to 5 parts by weight of a thickening agent; 0.8 to 1.5 part by weight of an isotonic agent; 0.001 to 0.05 part by weight of a bacteriostatic agent; 0.001 to 0.005 part by weight of a pH-adjusting agent; and 0.2 to 1 part by weight of a humectant.

The DHEA-based gel was prepared through the following method.

In step S1, 1 part by weight of the thickening agent was weighed and dissolved in 5 parts by weight of sterile water for injection, and then a pH was adjusted with the pH-adjusting agent to 6.5 to 8.0 to produce a first solution.

In step S2, 0.5 part by weight of a DHEA, 0.8 part by weight of the isotonic agent, 0.001 part by weight of the bacteriostatic agent, and 0.2 part by weight of the humectant were weighed and added to the solution 1, and sterile water for injection was supplemented until a resulting solution was of 100 parts by weight in total. The solution was thoroughly mixed, adjusted to a pH of 7, and filtered through a 0.22 μm microporous filter membrane for sterilization to produce the DHEA-based gel.

In this example, the thickening agent was carbomer, the isotonic agent was sodium chloride, the bacteriostatic agent was benzalkonium bromide, the pH-adjusting agent was phosphoric acid, and the humectant was sodium hyaluronate.

A mass percentage of DHEA in the DHEA-based gel obtained in this example was 0.5%.

### Test

### 1. Stability Investigation

In order to investigate the stability of the DHEA-based eye drop, eye ointment, and gel formulations of the present disclosure, 10 samples were collected through random sampling from each of the DHEA-based eye drops, the DHEA-based eye ointment, and the DHEA-based gel obtained in Examples 1, 3, and 4. Contents of DHEA in the eye drops, eye ointment, and gel were tracked and detected by the high-performance liquid chromatography-external standard method to investigate the stability. Specifically, experimental investigation conditions were as follows: temperature: 40° C.±2° C., and relative humidity: 75%+5%. Concentrations of the DHEA-based eye drops in Example 1 were 0.01%, 0.05%, 0.1%, 0.5%, 1%, and 2%, respectively.

The stability of the DHEA-based ophthalmic formulations was investigated by detecting changes in DHEA contents. When a DHEA content changed by 5% or more, it indicated the start of a change. When a DHEA content changed by 10% or more, it indicated the ineffectiveness.

As a result, after 3 months of an accelerated test at 40° C., the DHEA-based eye drops, the DHEA-based eye ointment, and the DHEA-based gel in Examples 1, 3, and 4 all did not change significantly during an experimental cycle, and the appearances, labeled amounts, pH values, and sterility test results of the drugs all were qualified. Therefore, the DHEA-based eye drops, the DHEA-based eye ointment, and the DHEA-based gel of the present disclosure exhibited qualified stability.

### 2. Eye Irritation Test

New Zealand white rabbits were selected as test subjects and randomly divided into 8 groups with 3 rabbits in each group. There were no significant differences in the age and body weight among rabbits in the groups. There was no abnormality in the ophthalmic examination under a slit lamp before administration.

0.01%, 0.05%, 0.1%, 0.5%, 1%, and 2% DHEA-based eye drops were dropped to the conjunctival sacs of the left eyes of white rabbits in groups 1 to 6 at an amount of 0.05 mL, respectively. 0.1 g of the DHEA-based eye ointment in Example 3 was dropped to the conjunctival sac of the left eye of each white rabbit in group 7. 0.1 g of the DHEA-based gel in Example 4 was dropped to the conjunctival sac of the left eye of each white rabbit in group 8. The right eye of each white rabbit was taken as a control group thereof, and the same amount of a 0.9% sodium chloride solution was dropped to the right eye.

In the groups 1 to 6, the DHEA-based eye drops each were dropped 4 times a day consecutively for 7 days. In the group 7, the DHEA-based eye ointment was dropped once a day consecutively for 7 days. In the group 8, the DHEA-based gel was dropped twice a day consecutively for 7 days. Local reactions were observed and recorded 1 hour after the first eye drop administration and before each administration. The blind trial was adopted, that is, observers did not know the grouping of the observed animals.

Results were scored based on the evaluation criteria for ophthalmic irritation responses in the Guidelines for Preclinical Research of New Drugs (Western Medicines) (Table 3 below).

TABLE 3
Scoring criteria

| Item | Ophthalmic irritation responses | Score |
|---|---|---|
| Corneal opacity | No opacity | 0 |
| (based on | There is scattered or diffused | 1 |
| the densest | opacity, and the iris is clearly visible | |
| part) | The translucent zone is recognizable, | 2 |
| | and the iris is blurred | |
| | There is a grey-white translucent | 3 |
| | zone, the iris details are blurred, and | |
| | the pupil size is barely clear | |
| | The cornea is opaque, and the iris | 4 |
| | is unrecognizable due to opacity | |
| Iris | Normal | 0 |
| | The folds are markedly deepened, | 1 |
| | there are congestion and swelling, there | |
| | is mild congestion around the cornea, and | |
| | the pupil is still responsive to light | |
| | There are bleeding and necrosis | 2 |
| | visible to the naked eyes, and there is no | |
| | response to light (or one of them) | |
| Conjunctiva | The blood vessels are normal | 0 |
| A. Congestion | The blood vessels are congested | 1 |
| | and bright-red | |
| | The blood vessels are congested and | 2 |
| | dark-red, and are not easily distinguished | |
| | The blood vessels are diffusely | 3 |
| | congested and purple-red | |
| Conjunctiva | No edema | 0 |
| B. Edema | Slight edema (involving the | 1 |
| | nictitating membrane) | |
| | Obvious edema accompanied | 2 |
| | by the partial ectropion | |
| | Edema to make nearly a half | 3 |
| | of the eyelid closed | |
| | Edema to make more than a | 4 |
| | half the eyelid closed | |
| Conjunctiva | No secretion | 0 |
| C. secretion | Small amount of secretions | 1 |
| | The secretions make the eyelid | 2 |
| | and eyelash moist or sticky | |
| | The secretions make the entire | 3 |
| | eye zone moist or sticky | |
| Total score | | 16 |

According to a total score, a corresponding irritation degree was determined. Evaluation criteria for eye irritation were shown in Table 4 below.

TABLE 4
Evaluation criteria for eye irritation

| Irritation degree | Score |
|---|---|
| No irritation | 0-3 |
| Mild irritation | 4-8 |
| Moderate irritation | 9-12 |
| Intense irritation | 13-16 |

Test results: Observation results of ophthalmic irritation in each group 1 hour and 7 days after the first eye drop administration were shown in Table 5.

TABLE 5
Observation results of ophthalmic irritation

| | Score at 1 hour after | |
|---|---|---|
| | the first eye | |
| Group | drop application | Score on day 7 |
| 0.9% sodium chloride | 1 | 2 |
| solution | | |
| 0.01% DHEA-based eye | 1 | 2 |
| drop | | |
| 0.05% DHEA-based eye | 1 | 2 |
| drop | | |
| 0.1% DHEA-based eye drop | 1 | 3 |
| 0.5% DHEA-based eye drop | 1 | 3 |
| 1% DHEA-based eye drop | 1 | 3 |
| 2% DHEA-based eye drop | 1 | 3 |
| DHEA-based eye ointment | 1 | 3 |
| DHEA-based gel | 1 | 3 |

The results showed that the DHEA-based eye drops of different concentrations, the DHEA-based eye ointment, and the DHEA-based gel in the experimental groups exhibited comparable irritation to the normal saline group, indicating that the DHEA-based eye drops of the technical solution have small ophthalmic irritation and excellent tolerance.

### 3. Experimental Research on Toxicology

Acute toxicity test is as follows. Healthy New Zealand white rabbits of half male and half female were selected with the inclusion criteria: no external eye diseases and normal reflection of pupils in both eyes to light. The white rabbits were randomly divided into 4 groups with 6 animals in each group, with groups 1 to 3 being the administration groups, and a group 4 being a blank control group. In the blank control group, normal saline was applied. In the administration groups, the 2% DHEA-based eye drop in Example 1, the DHEA-based eye ointment in Example 3, and the DHEA-based gel in Example 4 of the present disclosure were applied, respectively. A dose was 25 times an intended daily dose for adults. When observed for 7 days, the animals did not undergo abnormal changes, indicating that the administration of the drugs was safe.

### 4. Efficacy Research

(1) A total of 40 healthy three-week-old colored guinea pigs were randomly selected and randomly divided into 8 groups with 5 guinea pigs in each group. The 8 guinea pig groups includes a first group: a normal control group, a second group: a myopia model-alone group, a third group: a myopia model+0.01% DHEA-based eye drop group, a fourth group: a myopia model+0.1% DHEA-based eye drop group, a fifth group: myopia model+1% DHEA-based eye drop group, a sixth group: a myopia model+2% DHEA-based eye drop group, a seventh group: a myopia model+DHEA-based eye ointment group, and an eighth group: a myopia model+DHEA-based gel group. There were no statistically-significant differences in the baseline diopter and AL of the right eye among the groups.

(2) Establishment of a form deprivation myopia (FDM) model: the right eye was covered and the left eye was normally opened. The daily observation was conducted to ensure the covering effect for the right eye until the experiment was completed 4 weeks later. For guinea pigs in the third to eighth groups, the left eye was normally opened and the right eye was covered (FDM).

(3) Administration scheme: for guinea pigs in the third to sixth groups, 0.01%, 0.1%, 1%, and 2% DHEA-based eye drops were applied to the right eye at a dose of 50 μL four times a day consecutively for 7 days, respectively; for guinea pigs in the seventh group, the DHEA-based eye ointment in Example 3 was applied to the right eye at a dose of 0.1 g once a day consecutively for 7 days; and for guinea pigs in the eighth group, the DHEA-based gel in Example 4 was applied to the right eye at a dose of 0.1 g twice a day consecutively for 7 days.

(4) Measurement of myopia parameters:

A) Diopter measurement: the optometry was conducted with a streak retinoscope and lenses of different diopters, and an average of diopters in horizontal and vertical meridians was taken. The optometry was conducted 3 times, and an average was taken and recorded as a result.

B) AL measurement: ALs of both eyes were measured by an A-mode ultrasound instrument. Results of 6 measurements under waveform standards were selected, and an average was taken and recorded as a result, which was accurate to 0.01 mm.

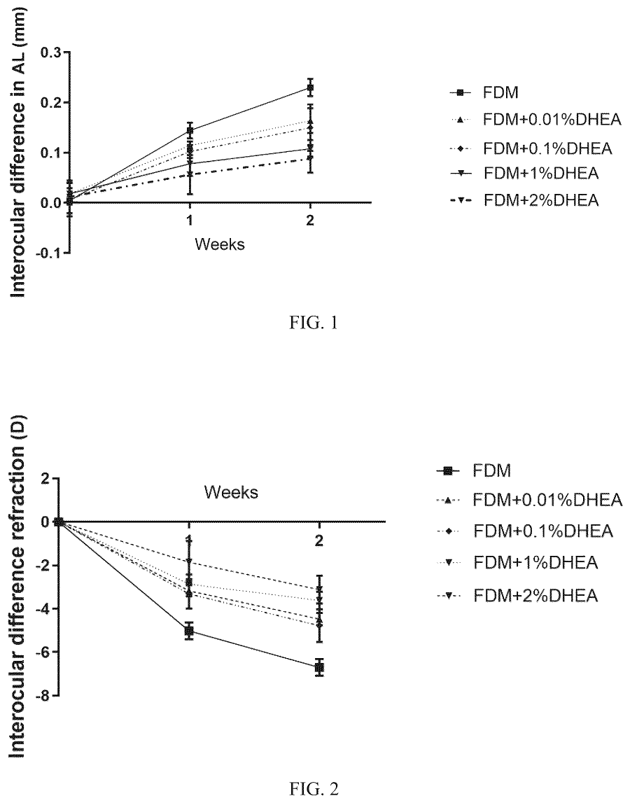

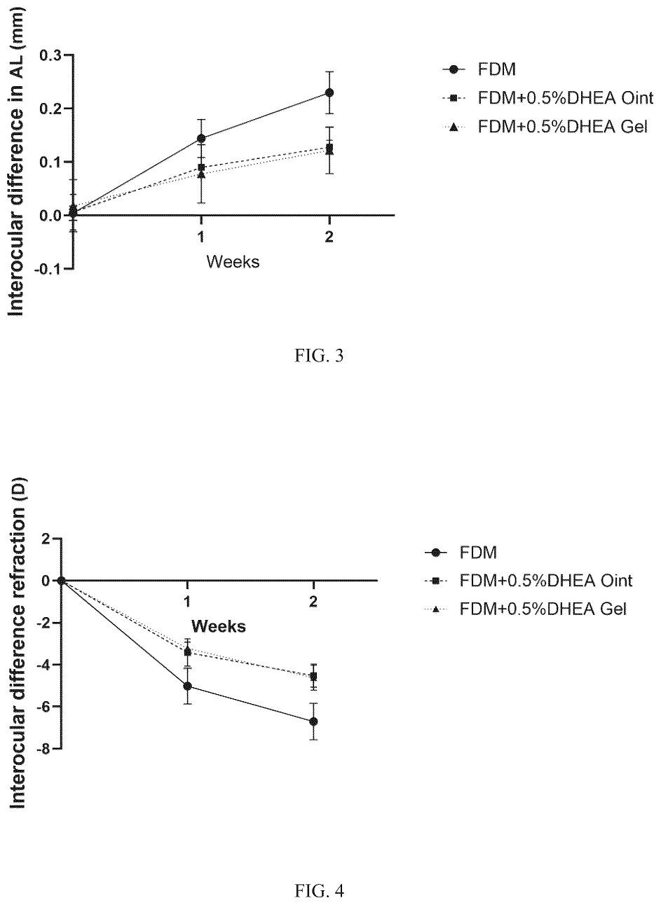

(5) Results: the experimental results were shown in FIG. 1 to FIG. 4. FIG. 1 shows the changes in ALs after the application of DHEA-based eye drops. FIG. 2 shows the changes in diopters after the application of DHEA-based eye drops. FIG. 3 shows the changes in ALs after the application of the DHEA-based eye ointment (Oint) and a DHEA-based gel (Gel). FIG. 4 shows the changes in diopters after the application of a DHEA-based eye ointment (Oint) and a DHEA-based gel (Gel). In the figures, p<0.05.

It can be seen from FIG. 1 that, after the experimental eyes were covered for 2 weeks, the growth in AL of each of the FDM+0.01% DHEA-based eye drop group, the FDM+0.1% DHEA-based eye drop group, the FDM+1% DHEA-based eye drop group, and the FDM+2% DHEA-based eye drop group was significantly slowed down compared with the myopia model group, and there were statistical differences.

It can be seen from FIG. 2 that, after the experimental eyes were covered for 2 weeks, the diopters of the FDM+0.01% DHEA-based eye drop group, the FDM+0.1% DHEA-based eye drop group, the FDM+1% DHEA-based eye drop group, and the FDM+2% DHEA-based eye drop group were significantly reduced compared with the myopia model group, and there were statistical differences.

It can be seen from FIG. 3 that, after the experimental eyes were covered for 2 weeks, the growth in AL of each of the DHEA-based gel group and the DHEA-based eye ointment group was also significantly slowed down compared with the myopia model group, and there were statistical differences.

It can be seen from FIG. 4 that, after the experimental eyes were covered for 2 weeks, the diopters of the DHEA-based gel group and the DHEA-based eye ointment group were also significantly reduced compared with the myopia model group, and there were statistical differences.

Therefore, the DHEA-based eye drop, the DHEA-based gel, and the DHEA-based eye ointment of the present disclosure all have the beneficial effect of preventing and controlling myopia.

Apparently, the above embodiments of the present disclosure are merely examples listed to clearly illustrate the technical solutions of the present disclosure, and are not intended to limit the specific implementations of the present disclosure. Any modifications, equivalent substitutions, improvements, etc. made within the spirit and principle of the claims of the present disclosure should be included within the protection scope of the claims of the present disclosure.

What is claimed is:

1. Use of dehydroepiandrosterone (DHEA) in preparation of a drug for preventing and treating ophthalmic myopia.

2. The use according to claim 1, wherein the drug is for external use.

3. The use according to claim 1, wherein a dosage form of the drug is an eye drop, an eye ointment, or a gel.

4. A DHEA-based eye drop prepared according to claim 1, comprising the following pharmaceutical components in parts by weight:
hydroxypropyl methylcellulose: 8 to 10 parts;
boric acid: 24 to 28 parts;
borax: 1 to 5 parts;
benzalkonium chloride: 0.05 to 0.1 part;
DHEA: 0.04 to 8 parts;
a pH-adjusting agent: 0.001 to 0.1 part; and
an osmolarity adjusting agent: 0.001 to 0.1 part.

5. A preparation method of the DHEA-based eye drop according to claim 4, comprising the following steps of:
S1) weighing 8 to 10 parts by weight of the hydroxypropyl methylcellulose, 24 to 28 parts by weight of the boric acid, 1 to 5 parts by weight of the borax, 0.05 to 0.1 part by weight of the benzalkonium chloride, and 1,350 to 2,150 parts by weight of purified water to prepare a blank solvent;
S2) weighing 0.04 to 8 parts by weight of the DHEA and 400 parts by weight of the blank solvent to prepare a DHEA solution; and
S3) adding the pH-adjusting agent and the osmolarity adjusting agent to the DHEA solution, respectively, to adjust a pH and an osmolarity of the DHEA solution to 6.2 to 7.2 and 280 mOsm/L to 310 mOsm/L respectively to produce the DHEA-based eye drop.

6. The preparation method of the DHEA-based eye drop according to claim 5, wherein in the step of S1, the blank solvent is prepared through the following steps of:
S11) weighing 8 to 10 parts by weight of the hydroxypropyl methylcellulose, and dissolving the hydroxypropyl methylcellulose in 350 to 450 parts by weight of the purified water to produce a first solution;
S12) weighing 24 parts to 28 parts by weight of the boric acid and 1 to 5 parts by weight of the borax, and dissolving the boric acid and the borax in the first solution to produce a second solution; and
S13) weighing 0.05 to 0.1 part by weight of the benzalkonium chloride, dissolving the benzalkonium chloride in the second solution, and diluting with 1,300 to 1,700 parts by weight of the purified water to a specified volume or concentration to produce the blank solvent; and

7. The preparation method of the DHEA-based eye drop according to claim 6, wherein in the step of S2, the DHEA solution is prepared through the following steps of:
weighing 0.04 to 8 parts by weight of the DHEA and 400 parts by weight of the blank solvent; and
dispersing the DHEA evenly in the blank solvent with a high-shear emulsifying machine to produce the DHEA solution.

8. A DHEA-based eye ointment prepared according to claim 1, comprising the following pharmaceutical components in parts by weight:
DHEA: 5 to 10 parts;
lanolin: 25 to 50 parts;
sterile liquid paraffin: 12 to 25 parts; and
yellow Vaseline: 850 to 1,000 parts.

9. A preparation method of the DHEA-based eye ointment according to claim 8, comprising the following steps of:
S1) preparation of an eye ointment matrix, comprising:
weighing 25 to 50 parts by weight of the lanolin and 850 to 1,000 parts by weight of the yellow Vaseline, mixing the lanolin and the yellow Vaseline, and heating for melting; and then adding 12 to 25 parts by weight of the sterile liquid paraffin, thoroughly mixing, and cooling to produce the eye ointment matrix;
S2) weighing 5 to 10 parts by weight of the DHEA, and dissolving the DHEA in 4 to 8 parts by weight of sterile water for injection to produce a mixed solution; and
S3) adding the mixed solution obtained in the step S2 to the eye ointment matrix obtained in the step S1, and thoroughly stirring to produce the DHEA-based eye ointment.

10. A DHEA-based gel prepared according to claim 1, comprising the following pharmaceutical components in parts by weight:
DHEA: 0.5 to 2 parts,
a thickening agent: 1 to 5 parts;
an isotonic agent: 0.8 to 1.5 part;
a bacteriostatic agent: 0.001 to 0.05 part;
a pH-adjusting agent: 0.001 to 0.005 part; and
a humectant: 0.2 to 1 part.

11. A preparation method of the DHEA-based gel according to claim 10, comprising the following steps of:
S1) weighing 1 to 5 parts by weight of the thickening agent, dissolving the thickening agent in 5 to 10 parts by weight of sterile water for injection, and then adjusting a pH with the pH-adjusting agent to 6.5 to 8.0 to produce a first solution; and
S2) weighing and adding 0.5 to 2 parts by weight of the DHEA, 0.8 to 1.5 parts by weight of the isotonic agent, 0.001 to 0.05 part by weight of the bacteriostatic agent, and 0.2 to 1 part by weight of the humectant to the first solution, supplementing sterile water for injection until a resulting solution is of 100 parts by weight in total, and thoroughly mixing to produce the DHEA-based gel.

12. Use of the DHEA-based eye drop according to claim 4 in preparation of a drug for preventing and treating ophthalmic myopia.

13. The use according to claim 12, wherein the ophthalmic myopia comprises true myopia and pseudomyopia.

14. Use of the DHEA-based eye ointment according to claim 8 in preparation of a drug for preventing and treating ophthalmic myopia.

15. The use according to claim 14, wherein the ophthalmic myopia comprises true myopia and pseudomyopia.

16. Use of the DHEA-based gel according to claim 10 in preparation of a drug for preventing and treating ophthalmic myopia.

17. The use according to claim 16, wherein the ophthalmic myopia comprises true myopia and pseudomyopia.
